(19) United States
(12) Patent Application Publication
(10) Pub. No.: US 2025/0134411 A1
(43) Pub. Date: May 1, 2025

(54) APPARATUS, METHOD, COMPUTER PROGRAM AND SYSTEM FOR DETERMINING A VITAL SIGN

(71) Applicant: Sony Semiconductor Solutions Corporation, Kanagawa (JP)

(72) Inventor: Diederik Paul MOEYS, Zurich (CH)

(73) Assignee: Sony Semiconductor Solutions Corporation, Kanagawa (JP)

(21) Appl. No.: 18/834,645

(22) PCT Filed: Feb. 20, 2023
(86) PCT No.: PCT/EP2023/054181

§ 371 Date: Jul. 31, 2024

(30) Foreign Application Priority Data
Feb. 24, 2022 (EP) 22158599.5

(51) Int. Cl. A61B 5/11; A61B 5/00

(52) U.S. Cl. CPC A61B 5/1114; A61B 5/0077; A61B 5/7282

(57) ABSTRACT

Examples relate to an apparatus, method, computer program, and system for determining a vital sign of a living body based on motion determined from an event signal of an event-based vision sensor. The apparatus comprises an interface for communicating with an event-based vision sensor. The apparatus comprises processing circuitry configured to obtain an event signal from the event-based vision sensor, the event signal representing a detected change in luminance detected by the event-based vision sensor, determine a motion of at least a body part of a living body based on the event signal, and determine at least one vital sign of the living body based on the motion.

### FIELD

Examples relate to an apparatus, method, computer program, and system for determining a vital sign of a living body based on motion determined from an event signal of an event-based vision sensor.

### BACKGROUND

In hospitals and care homes, systems of continuous health monitoring are often either invasive (e.g., if a sensor is used that is directly in touch with the patient body), or computationally expensive (e.g., if the continuous health monitoring is vision-based). In the latter case, having a frame-based camera constantly recording the patient entails generating a lot of data, at a fixed, non-adaptable sampling rate. This may lead to elevated post-processing costs to extract dynamic information from all the frames and therefore to increased power consumption. Frame-based cameras also suffer from limited dynamic range and motion blur. Finally, cameras are also a privacy concern.

In a research environment or surgery setting, recording microscopic, low-contrast occurrences at high frame rate and high resolution with regular CMOS frame-based sensors can be expensive in terms of data rate and size, power consumption and postprocessing.

There may be a desire for an improved concept for the monitoring of patients.

### SUMMARY

This desire is addressed by the subject-matter of the independent claims.

Various aspects of the present disclosure are based on the finding that some types of vital signs of a person are perceptible through the motion generated by the respective vital sign. For example, the breathing of a person is perceptible via the chest of the person moving, and the heart rate of a person is perceptible via a pulsating motion where the blood vessels are close to the skin. Even minute amounts of motion, such as motion caused by breathing or the heart rate, cause changes in the way light is reflected off the person. So-called event-based vision sensors (EVS) can be used to detect changes in luminance that are caused by the motion of the person, while overcoming many drawbacks of frame-based sensors. Event-based vision sensors generate event signals, which are signals that represent the change in luminance detected by the EVS without being tied to a fixed frame rate. When a suitable contrast threshold is used for generating events for the event signal, the resulting low-noise event signal can be analyzed with a reduced effort compared to the video signals of frame-based cameras to determine the motion, and thus the vital sign, of the person.

Various examples of the present disclosure relate to an apparatus. The apparatus comprises an interface for communicating with an event-based vision sensor. The apparatus comprises processing circuitry configured to obtain an event signal from the event-based vision sensor. The event signal represents a detected change in luminance detected by the event-based vision sensor. The processing circuitry is configured to determine a motion of at least a body part of a living body based on the event signal. The processing circuitry is configured to determine at least one vital sign of the living body based on the motion. Thus, an improved concept for the monitoring of patients is provided.

In general, vital signs of living bodies, e.g., of persons, are repetitive. For example, the heart of a person beats approximately 40 to 150 times per minute, and the person takes 10 to 30 breaths per minute. The vital signs of a person thus causes repetitive motion, and in particular periodic motion. Accordingly, the processing circuitry may be configured to determine the at least one vital sign of the living body based on a periodicity of the motion. For example, the processing circuitry may be configured to determine at least one of a heart rate of the living body and a breathing rate of the living body based on the (periodic) motion.

Such periodic motion may be determined by analyzing the event signal. The processing circuitry may be configured to determine peaks in the event signal. The processing circuitry may be configured to determine the periodicity of the motion based on the peaks in the event signal. The focus on the peaks may be used to disregard a low-level noise in the event signal. Moreover, the peaks may be analyzed to determine which of the peaks relate to periodic and which relate to non-periodic motion. For example, the processing circuitry may be configured to determine the at least one vital sign based on periodic motion exhibited by the living body.

In effect, the processing circuitry may be configured to disregard non-periodic motion represented in the event signal.

Such non-periodic motion may also occur when the living body, e.g., the person, moves consciously or sub-consciously, e.g., when the living body turns from one side to the other during sleep. The processing circuitry may be configured to disregard motion having an extent that exceeds a motion threshold, e.g., motion having an extent that is larger than the extent commonly related to the vital sign.

As outlined above, vital signs are often observed as periodic events, which are caused by periodic motion. The processing circuitry may be configured to determine the periodic motion based on the peaks in the event signal and based on a pre-determined range of supported periodicity. The pre-determined range of supported periodicity may be based on the vital sign being determined. For example, the pre-determined range of supported periodicity may impose a lower limit and an upper limit of periodicities that are characteristic for the vital sign being determined. This may limit the search space during the identification of the periodic motion.

In the present case, the event signal is used to determine small-scale motion of individual portions of the body. If the event signal is aggregated, the distribution of the motion can be used to derive an outline of the body, as most body-parts generally move at some point or other. The processing circuitry may be configured to aggregate the event signal over a predefined time interval, and to determine an outline of the living body based on the aggregated event signal. The processing circuitry may thus be configured to determine the at least one vital sign based on the outline of the living body. For example, luminance changes, and thus motion, that occur outside the outline, can be disregarded, as they are likely not caused by the body. Accordingly, the processing circuitry is configured to disregard a portion of the event signal based on the outline of the living body.

Additionally, or alternatively, the outline may be used to determine the identity of the living body. For example, the living body may be a living human body. The processing circuitry may be configured to process the outline of the living body using a machine-learning model to determine the identity of the living human body.

For example, the processing circuitry may be configured to determine the at least one vital sign based on the identity of the living human body. In particular, the processing circuitry may be configured to determine a pre-determined range of supported periodicity of the motion based on the identity of the living human body. This may further limit the search space for the determination of the periodic motion, and thus vital signs.

To limit the noise present in the event signal, the so-called contrast threshold of the event-based vision sensor may be adjusted to the extent of the periodic motion caused by the vital signs. For example, the processing circuitry may be configured to determine peaks in the event signal, and to adjust a contrast threshold of the event-based vision sensor based on a ratio between the peaks in the event signal and other portions of the event signal. For example, the peaks may be (predominately) caused by the periodic motion indicating the vital signs.

In various examples, different contrast thresholds may be evaluated until one is determined that exhibits desired properties. For example, the processing circuitry may be configured to sweep the contrast threshold, determine peaks in the event signal that are based on the sweep of the contrast threshold, and set a contrast threshold that yields a desired ratio between the peaks in the event signal and other portions of the event signal.

The determined at least one vital sign may subsequently be used for various purposes. For example, the processing circuitry may be configured to log the at least one vital sign based on the identity of the living human body. This may enable a subsequent analysis of the vital signs, e.g., to detect long-term changes or to correlate variations in the vital signs with the use of specific medication.

Alternatively, or additionally, some information on the vital signs may be provided to another entity, e.g., to a monitoring system, an alarm system, or to a medical intervention device. For example, the processing circuitry may be configured to generate an output signal based on the determined at least one vital sign.

In many cases, as long as the vital signs are in a “healthy” range, no action may be required. Accordingly, the processing circuitry is configured to generate the output signal if the determined at least one vital sign exceeds an upper threshold or falls below a lower threshold. For example, the threshold(s) may be chosen such, that the output signal is only provided in an emergency or pre-emergency situation.

In hospitals or care homes, when the vital signs are outside a pre-defined range that is deemed healthy, the personnel may be alerted of the potentially life-threatening condition of the living body. For example, the output signal comprises an alarm signal, which may be used to alert the care personnel.

In some examples, countermeasures may be taken when an emergency situation occurs. For example, the processing circuitry may be configured to control a medical intervention device, such as a defibrillation device or a device for controlling a release of medication, using the output signal. This may help stabilize the living body until personnel can arrive.

Various examples of the present disclosure relate to a corresponding (computer-implemented) method. The method comprises obtaining an event signal from an event-based vision sensor. The event signal represents a detected change in luminance detected by the event-based vision sensor. The method comprises determining a motion of at least a body part of a living body based on the event signal. The method comprises determining at least one vital sign of the living body based on the motion.

Various examples of the present disclosure relate to a corresponding computer program with a program code for performing the above method when the computer program is executed on a processor.

Various examples of the present disclosure relate to a system comprising a camera with an event-based vision sensor and the apparatus introduced above.

For example, the camera may be one of a wall-mounted camera, a ceiling-mounted camera, and a wearable camera. Wall-mounted cameras and ceiling-mounted cameras can be mounted in hospital or care facility rooms and may be used to provide less-invasive monitoring of the vital signs of the occupants. A wearable camera may be worn around the wrist or as collar, e.g., for determining the heart rate.

In some example, the system may further comprise a medical intervention device, with the apparatus being configured to control the medical intervention device. The medical intervention device may help stabilize the living body until personnel can arrive.

### BRIEF DESCRIPTION OF THE FIGURES

Some examples of apparatuses and/or methods will be described in the following by way of example only, and with reference to the accompanying figures, in which

FIG. 1a shows a block diagram of an example of an apparatus for determining at least one vital sign of a living body;

FIG. 1b shows a schematic diagram of an example of a system comprising a camera with an event-based vision sensor and an apparatus for determining at least one vital sign of a living body;

FIG. 2 shows a flow chart of an example of a method for determining at least one vital sign of a living body;

FIG. 3 shows a flow chart of an example of an algorithm for monitoring vital signs;

FIG. 4 shows a schematic drawing of the application of the proposed algorithm in a care home or hospital;

FIG. 5 shows a flow chart of an example of an algorithm for initial calibration;

FIG. 6 shows a flow chart of an example of an algorithm for microscopic patient monitoring; and

FIG. 7 shows a schematic diagram of microscopic monitoring of blood cells in tissue.

### DETAILED DESCRIPTION

Some examples are now described in more detail with reference to the enclosed figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an “or”, this is to be understood as disclosing all possible combinations, i.e., only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

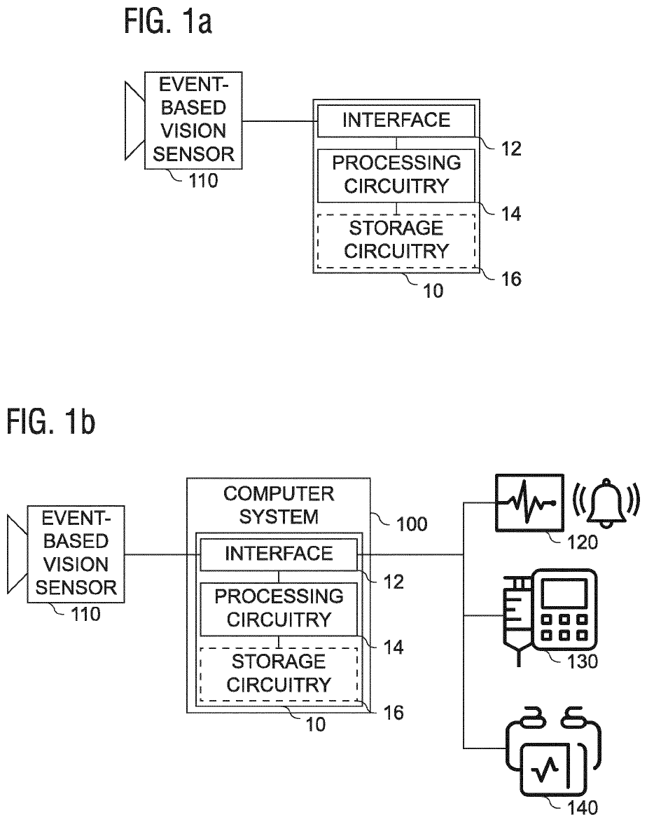

FIG. 1a shows a block diagram of an example of an apparatus 10 for determining at least one vital sign of a living body. The apparatus 10 comprises an interface 12 and processing circuitry 14. Optionally, the apparatus 10 further comprises storage circuitry 16. The processing circuitry 14 is coupled to the interface 12 and the (optional) storage circuitry 16. In general, the functionality of the apparatus 10 is provided by the processing circuitry 14, in conjunction with the interface 12 (for communicating with other entities and devices, such as an event-based vision sensor 110, and/or, as shown in FIG. 1b, a medical monitor 120, an infusion pump 130, or a defibrillation device 140) and/or the optional storage circuitry 16 (for storing information, such as an aggregated event signal).

The processing circuitry 14 is configured to obtain an event signal from the event-based vision sensor 110. The event signal represents a detected change in luminance detected by the event-based vision sensor. The processing circuitry 14 is configured to determine a motion of at least a body part of a living body based on the event signal. The processing circuitry 14 is configured to determine at least one vital sign of the living body based on the motion.

The apparatus 10 is part of a system of devices comprising the apparatus 10, the event-based vision sensor, and, optionally, one or more additional devices. FIG. 1b shows a schematic diagram of an example of a system comprising a camera with the event-based vision sensor 110, the apparatus 10 and one or more optional additional devices, such as the medical monitor 120, the infusion pump 130, or the defibrillation device 140). The event-based vision sensor 110 and the optional medical monitor 120, infusion pump 130, and defibrillation device 140 are coupled with the apparatus 10 via the interface 12. As shown in FIG. 1b, the apparatus 10 may be part of, e.g., implemented by, a computer system 100.

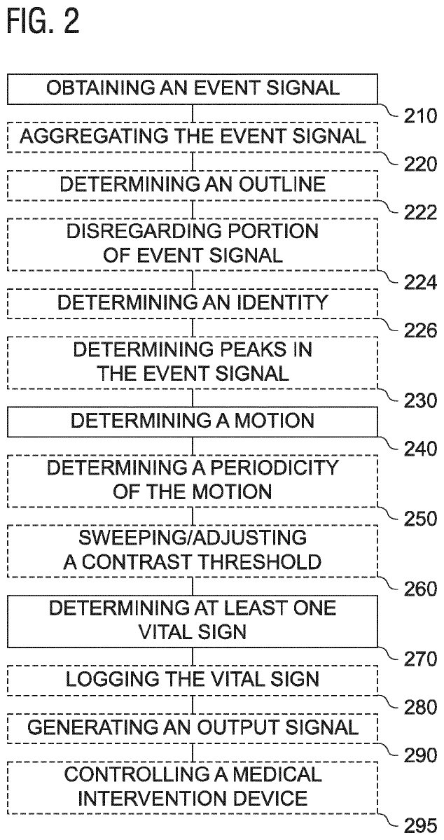

In FIGS. 1a and 1b, the proposed concept is illustrated with respect to an apparatus 10 and a system comprising said apparatus. In some examples, the proposed concept may be embodied by a corresponding method, which is illustrated in connection with FIG. 2. FIG. 2 shows a flow chart of an example of a corresponding (computer-implemented) method for determining at least one vital sign of a living body. The method comprises obtaining 210 the event signal from the event-based vision sensor. The method comprises determining the motion of at least the body part of the living body based on the event signal. The method comprises determining 270 the at least one vital sign of the living body based on the motion. For example, the method may be performed by the apparatus 10 or computer system 100 shown in FIGS. 1a and/or 1b.

In the following, the features of the apparatus 10, of the system, of the corresponding method, and of a corresponding computer program, are illustrated with respect to the apparatus 10 and system comprising said apparatus. Features introduced in connection with the apparatus 10 and system comprising said apparatus 10 may likewise be introduced in the corresponding method and computer program.

The proposed concept relates to an approach to determining at least one vital sign of a living body with the help of an event-based vision sensor. Event-based vision sensors, such as the event-based vision sensor 110, are also referred to as event cameras or dynamic vision sensors. They are bio-inspired sensors that differ from conventional frame cameras: Instead of capturing images at a fixed rate, they may asynchronously measure brightness changes, and output a stream of events, e.g., the event signal, that encode the time and sign or amplitude of the brightness changes. In some examples, the event-based vision sensor 110 may comprise a single photo-sensitive element (i.e., “pixel”) for measuring the brightness changes. Alternatively, the event-based vision sensor 110 may comprise a plurality of pixels, which may be arranged in a two-dimensional pixel array. In this case, the event signal may encode per-pixel brightness changes, and include the time, location and sign/amplitude of the brightness changes. Pixels in the event-based vision sensor 110 may work independently and asynchronously. Whenever one of the pixels observes a significant change in intensity, it may output an ON or OFF event along with the position of the sensing pixel (in case multiple pixels are used). An ON event stands for sensing an increasing change of luminance, whereas an OFF event stands for a decreasing change. Those pixels without significant change of intensity might trigger no output at all.

In various examples of the present disclosure, the event-based vision sensor 110 is part of a camera that is used to monitor the living body. In particular, such a camera may be used to monitor human living bodies, i.e., persons, e.g., in hospitals or care facilities. In other words, the living body may be a person, e.g., a person being monitored in a hospital or a care facility (or even at home, in sleep apnea monitoring or tachycardia monitoring). Alternatively, the living body may be an animal, e.g., an animal in an animal clinic or in a stable. The living body, e.g., person or animal, is monitored using the camera. To limit the invasiveness of the monitoring, the camera may be kept in the background. For example, the camera may be mounted at a wall or ceiling. Accordingly, the camera may be a wall-mounted camera or a ceiling-mounted camera. Alternatively, the camera may be worn by the living body, e.g., person or animal, being monitored, e.g., as part of a wristband or collar, with the camera being directed towards the skin of the living body. In another set of examples, the event-based vision sensor 110 may be part of a microscope, i.e., it may be coupled with a microscope lens. In these examples, the proposed concept may be used to determine oxygenation (e.g., brain oxygenation) from a blood flow.

The vital sign is determined by analyzing the event signal provided by the event-based vision sensor 110. Accordingly, the processing circuitry is configured to obtain (e.g., receive) the event signal from the event-based vision sensor, e.g., via a wired data connection, such as ethernet or the universal serial bus, or via a wireless data connection, such as Bluetooth, WiFi, or a proprietary wireless communication protocol. For example, the processing circuitry may be configured to receive the event signal in near-real time, e.g., with a maximal delay of 1 second (or 2 seconds, or 5 seconds) between an event being generated by the event-based vision sensor and the event being registered at the apparatus as part of the event signal. The processing circuitry 14 may thus also process the event signal in near-real time.

As outlined above, the event signal represents a detected change in luminance detected by the event-based vision sensor. The event signal comprises a sequence of events, which represent the changes of luminance detected by the event-based vision sensor. In various examples, each event comprises information on a sign and amplitude of the luminance changes, e.g., whether the luminance increases or decreases (i.e., the sign) and how large the change in the luminance is (i.e., the amplitude). In various examples of the present concept, each event further comprise information on a location (i.e., which pixel of the two-dimensional grid of pixels is affected) of the detected change of luminance. Whether an event is triggered and inserted into the event signal in response to a change in luminance is determined based on a so-called contrast threshold, which relates to the minimal contrast between two levels of luminance observed by the event-based vision sensors that triggers an event. Depending on the contrast threshold being used, a lower or higher change in luminance suffices for triggering an event, and thus more or fewer events are triggered.

The processing circuitry 14 is configured to determine the motion of at least the body part of the living body based on the event signal. In this context, it is assumed that a change in luminance can be caused by a motion of the living body. In other words, at least a subset of the events representing a change in luminance are caused by a motion of the living body. For example, the motion of the living body may be observable from outside the body. The processing circuitry 14 may thus be configured to determine the motion of at least the body part of the living body based on events comprised in the event signal that are caused by the motion of the living body. However, not every change in luminance might be caused by a corresponding motion of the living body. Moreover, not every change in luminance caused by the motion of the living body might be of interest for determining the at least one vital sign. In particular, the motion of interest in the present case is low-amplitude motion, e.g., motion that ranges from less than a millimeter (for the skin pulsating from the heartbeat) to at most 5-10 centimeters (for the chest expanding and contracting due to a breathing motion). Therefore, various measures may be taken to disregard noise, such as events that are unrelated to motion caused by vital signs, in the determination of the vital signs.

In general, the vital signs of a living body express themselves through periodic motion. For example, the breathing of the living body is expressed by the chest inflating and deflating. The heart rate is expressed by portions of the skin pulsating based on the pulses of blood provided by the heart of the living body. The motion caused by the at least one vital sign may have at least one of the following two properties—the motion may be periodic, i.e., occur according to a time-pattern that is inherent to the vital sign, and the amplitude of the motion, and thus amplitude of the events, caused by the vital sign may be in a range of amplitudes that are characteristic for the respective vital sign. Both properties are related and may be used to disregard a subset of events of the event signal.

In the proposed concept, the periodic motion caused by the vital signs is of interest, in order to establish both which events of the event signal are caused by the periodic motion, and also to determine the characteristic range of amplitudes. The periodic motion of interest is represented by (amplitude) peaks in the event signal. The processing circuitry may be configured to determine the (amplitude) peaks in the event signal, and to determine the motion of the living body based on the peaks in the event signal. Accordingly, the method may comprise determining 230 the peaks in the event signal and determining 240 the motion of the living body based on the peaks in the event signal. To isolate the peaks, the processing circuitry may be configured to remove events having an amplitude that is below a pre-defined or relative lower amplitude threshold. However, not every peak may be of interest. Some peaks may be caused by motion that is not directly caused by the at least one vital signs (e.g., a person turning in the bed), or not caused by motion at all (e.g., a brightly lit vehicle passing the vehicles). Such events may be excluded by removing events that have an amplitude that is too high. For example, the processing circuitry may be configured to determine a median amplitude of the peaks in the event signal, and to disregard events having an amplitude that exceeds the median amplitude by at least 25% (or at least 50%, or at least 100%). Moreover, the processing circuitry may be configured to determine the relative lower amplitude threshold based on the median amplitude of the peaks.

In various examples, the processing circuitry is configured to determine the periodicity of the motion based on the peaks in the event signal. Accordingly, the method of FIG. 2 may comprise determining 250 the periodicity of the motion based on the peaks in the event signal. This may be done by fitting the peaks in the event signal to a periodic sequence of peaks having a given periodicity. For example, the processing circuitry may be configured to compare the peaks to in the event signal to a plurality of sequences of peaks that are each associated with a periodicity, and to determine the periodicity based on a match between the peaks to in the event signal and a sequence of the plurality of sequences of peaks associated with the periodicity. For example, the processing circuitry may be configured to determine the periodicity based on the best match between the peaks to in the event signal and a sequence of the plurality of sequences of peaks. The plurality of sequences of peaks that are each associated with a periodicity may be obtained based on a pre-determined range of supported periodicity that is based on the vital sign being determined. In other words, the processing circuitry may be configured to determine the periodic motion based on the peaks in the event signal and based on a pre-determined range of supported periodicity, with the pre-determined range of supported periodicity being based on the vital sign being determined. For example, for the breathing rate, a periodicity of 10 to 30 movements (i.e., events or peaks) per minute (i.e., breaths per minute) may be used. For the heartbeat, a periodicity of 40 to 150 movements (i.e., events or peaks) per minute may be used. Accordingly, the respective sequences of peaks may comprise between 10 and 30 peaks per minute (for determining the periodicity of a breathing motion), or between 40 and 150 peaks per minute (for determining the periodicity of the heartbeat). Accordingly, if the vital sign being determined is the heart rate, 40 peaks per minute may be considered a lower limit of the range of supported periodicity, and 150 peaks per minute may be considered a higher limit of the range of supported periodicity. Similarly, if the vital sign being determined is the breathing rate, 10 peaks per minute may be considered the lower limit, and 30 peaks per minute may be considered the upper limit. In conclusion, the pre-determined range of supported periodicity may impose a lower limit and an upper limit of periodicities that are characteristic for the vital sign being determined.

In addition, the processing circuitry may be configured to count the peaks in the event signal, to determine a starting point for fitting/matching the event signal to the sequence of peaks, with the starting point being based on the number of peaks per pre-defined unit of time.

The determination of the periodicity of the motion has the purpose of identifying the portions of the event signal that are caused by the periodic motion related to the respective vital sign. Other motion, e.g., non-periodic motion, such as the living body turning in their sleep, may be subsequently disregarded. Accordingly, the processing circuitry may be configured to disregard non-periodic motion represented in the event signal. Such non-periodic, conscious (or at least sub-conscious) motion is also often larger than the periodic motion being caused by the respective vital sign. Accordingly, the processing circuitry may be configured to disregard motion having an extent that exceeds a motion threshold, e.g., by determining the median amplitude of the peaks in the event signal, and disregarding events having an amplitude that exceeds the median amplitude by at least 25% (or at least 50%, or at least 100%), which is likely caused by motion exceeding the motion threshold.

To further reduce noise in the event signal, the above-mentioned contrast threshold may be adapted to suppress low-level noise and/or to obtain a desired signal-to-noise ratio (by removing low-level noise and/or scaling the amplitude peaks). In particular, the contrast threshold of the event-based motion sensor may be adjusted based on the ratio between the amplitude peaks, and in particular the periodic amplitude peaks, and the amplitude of other non-periodic events in the event signal. In other words, the processing circuitry may be configured to determine peaks in the event signal, and to adjust the contrast threshold of the event-based vision sensor based on a ratio between the peaks in the event signal and other portions of the event signal. For example, the processing circuitry may try and evaluate (i.e., “sweep” various contrast thresholds until one of the contrast thresholds yields a desired ration between the (periodic) peaks and the non-periodic events in the event signal. Thus, the processing circuitry may be configured to sweep the contrast threshold (i.e., incrementally adjust within a predefined range of contrast thresholds), determine the (periodic) peaks in the event signal that are based on the sweep of the contrast threshold, and to set a contrast threshold that yields a desired ratio between the peaks in the event signal and other portions of the event signal. Accordingly, the method of FIG. 2 may comprise adjusting/sweeping 260 the contrast threshold. For example, the desired ratio may be a ratio that is suitable for fitting the periodic peaks to a sequence of peaks associated with a periodicity.

Another two approaches for improving the determination of the periodic motion are based on using the event signal in an unconventional way—in addition to evaluating the events of the event signal separately, the events can be aggregated (e.g., collected over a pre-defined time interval, such as 30 seconds, or a minute, or two minutes, or five minutes) to establish the outline (i.e., shape) of the living body. In other words, the processing circuitry is configured to aggregate the event signal over a pre-defined time interval, and to determine the outline of the living body based on the aggregated event signal. Accordingly, as shown in FIG. 2, the method may comprise aggregating 220 the event signal, and determining 222 the outline. As most body parts exhibit minute and un-conscious motions over time, they register as part of the event signal (due to a change in luminance caused by the motion), in contrast to fixed objects such as the furniture. This outline may then be used to improve the determination of the periodic motion, and eventually the determination of the at least one vital sign. In other words, the processing circuitry may be configured to determine the at least one vital sign based on the outline of the living body, e.g., to determine the periodic motion based on the outline of the living body.

As a first approach, events that originate from outside the outline of the living body (or outside a portion of the living body, such as the chest) may be disregarded, as they are likely not to be caused by motion related to the at least one vital sign. Accordingly, the processing circuitry may be configured to disregard a portion of the event signal based on the outline of the living body, e.g., events that originate from outside the outline (or outside a portion of the outline) of the living body. Similarly, the method may comprise disregarding 224 the portion of the event signal based on the outline of the living body.

Additionally, or alternatively, as a second approach, the identity of the living body may be determined based on its outline, and the identity may be used to tune the determination of the living body. For example, as outlined above, the living body may be a living human body (i.e., a person). The processing circuitry may be configured to process the outline of the living body using a machine-learning model to determine an identity of the living human body. Accordingly, the method of FIG. 2, may comprise determining 226 the identity of the living human body. For example, the machine-learning model may be trained, e.g., using supervised learning, to output information on an identity of a human body based on the outline of the human body. For example, the machine-learning model may be trained using labelled data, e.g., using a plurality of samples of outlines of living human bodies as training input samples, and a plurality of corresponding instances of information on the identity of the living human bodies shown in the plurality of samples of outlines of the living human bodies as desired output in the supervised learning-based training of the machine-learning model. For example, the processing circuitry may be configured to determine the at least one vital sign based on the identity of the living human body. In particular, the processing circuitry may be configured to determine the pre-determined range of supported periodicity of the motion based on the identity of the living human body. For example, each living human body (i.e., person) may be associated with a corresponding range of supported periodicity, which may be based on the resting heart rate and/or based on the average or median breathing rate of the respective living human body

As a third approach, additionally or alternatively, the determined outline may be used to determine whether the contrast threshold is suitable. For example, if the determined outlined is spurious, the contrast threshold may be deemed unsuitable, and the contrast threshold may be lowered. If the determined outline is dissimilar to an outline of a living body (as pieces of furniture are part of the outline, the contrast threshold may also be deemed unsuitable, and the contrast threshold may be increased.

Once the (periodic) motion is determined, it is used to determine the at least one vital sign, e.g., at least one of the heart rate of the living body and the breathing rate of the living body, of the living body. In particular, wherein the processing circuitry is configured to determine the at least one vital sign based on the periodic motion (that is caused by the respective vital sign) exhibited by the living body, e.g., the periodic pulsation of the skin for determining the heart rate, or the periodic expansion and contraction of the chest for determining the breathing rate. In effect, the processing circuitry may be configured to determine the at least one vital sign of the living body based on the periodicity of the motion, e.g., by deriving the respective vital sign from the periodicity (that is, in turn, associated with the sequence of peaks being matched to the event signal)

In some examples, the determined at least one vital sign may be determined over a stretch of time (e.g., days, weeks or even months), and may be stored for further analysis. For example, the processing circuitry may be configured to log the at least one vital sign, e.g., using a database. Accordingly, as shown in FIG. 2, the method may comprise logging the at least one vital sign. For example, the processing circuitry may be configured to log the at least one vital sign based on the identity of the living human body, e.g., based on the identity of the living human body determined via the outline of the living human body, or based on the identity associated with a room where the living body is recorded by the event-based vision sensor (e.g., in a care home, where each occupant has a separate room).

Apart from logging, e.g., instead of or in addition to logging, the at least one vital sign may be displayed via a medical monitor, such as the medical monitor 120 shown in FIG. 1b. For example, the processing circuitry may be configured to generate an output signal based on the determined at least one vital sign. Accordingly, as shown in FIG. 2, the method may comprise generating 290 the output signal. For example, the output signal may comprise information on the at least one vital sign, e.g., at least one numerical value representing the at least one vital sign or a binary or ternary value representing whether the at least one vital sign has violated (e.g., exceeded or fallen below) a lower/upper threshold. In the former case, the out-put signal may be provided in any case, e.g., for displaying via the medical monitor 120. In the latter case, the provision of the output signal may be made dependent on the at least one vital sign violating the upper or lower threshold. In other words, the processing circuitry may be configured to generate the output signal if the determined at least one vital sign exceeds the upper threshold or falls below the lower threshold.

In this case, at least one of the following two courses of actions can be taken—personnel may be alerted via an alarm signal, and counteractive measures may be taken. For example, the output signal may comprise an alarm signal, i.e., a signal that triggers an alarm signal at an output device, such as the medical monitor 120 (e.g., the “bell” shown in 120, which may be a digital alarm sound is commonly used in medical monitors).

Additionally, or alternatively, the processing circuitry may be configured to control a medical intervention device 130; 140 (that may be part of the signal shown in FIG. 1b) using the output signal. Accordingly, as shown in FIG. 2, the method may comprise controlling 295 the medical intervention device. For example, as shown in FIG. 1b, the medical intervention device may be a device 130 for controlling a release of medication, such as caffeine or, in more urgent cases, adrenaline to the living body (e.g., an infusion pump), or a defibrillation device (that may be worn by the respective human body).

In another set of examples, the proposed concept may be used for microscopic patient monitoring. In this case, the motion may relate to microscopic motion of blood cells within blood vessels (inside the living body), e.g., instead of motion of body parts that is visible from outside the body. For example, in this case, the vital sign being determined may be the oxygenation of an organ, such as the brain, which may be derived from the rate of blood flow determined from the event signal. As result of the blood flow (or oxygenation) monitoring, drugs such as vasodilator drugs or vasoconstrictor drugs may be released by the device 130 for controlling a release of medication.

Some examples of the present disclosure use machine learning to identify the human living body based on its outline. Machine learning refers to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and associated training content information, the machine-learning model “learns” to recognize the content of the images, so the content of images that are not included of the training images can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model.

Machine-learning models are trained using training input data. The examples specified above use a training method called “supervised learning”. In supervised learning, the machine-learning model is trained using a plurality of training samples, wherein each sample may comprise a plurality of input data values, and a plurality of desired output values, i.e., each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm, e.g., a classification algorithm, a regression algorithm, or a similarity learning algorithm. Classification algorithms may be used when the outputs are restricted to a limited set of values, i.e., the input is classified to one of the limited set of values. Regression algorithms may be used when the outputs may have any numerical value (within a range). Similarity learning algorithms are similar to both classification and regression algorithms but are based on learning from examples using a similarity function that measures how similar or related two objects are.

For example, the machine-learning model may be an artificial neural network (ANN). ANNs are systems that are inspired by biological neural networks, such as can be found in a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of the sum of its inputs. The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e., to achieve a desired output for a given input. In at least some embodiments, the machine-learning model may be deep neural network, e.g., a neural network comprising one or more layers of hidden nodes (i.e., hidden layers), prefer-ably a plurality of layers of hidden nodes.

The interface 12 may correspond to one or more inputs and/or outputs for receiving and/or transmitting information, which may be in digital (bit) values according to a specified code, within a module, between modules or between modules of different entities. For example, the interface 12 may comprise interface circuitry configured to receive and/or transmit information.

The processing circuitry 14 may be implemented using one or more processing units, one or more processing devices, any means for processing, such as a processor, a computer or a programmable hardware component being operable with accordingly adapted software. In other words, the described function of the processing circuitry 14 may as well be implemented in software, which is then executed on one or more programmable hardware components.

Such hardware components may comprise a general-purpose processor, a Digital Signal Processor (DSP), a micro-controller, etc.

In at least some embodiments, the storage circuitry 16 may comprise at least one element of the group of a computer readable storage medium, such as a magnetic or optical storage medium, e.g., a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

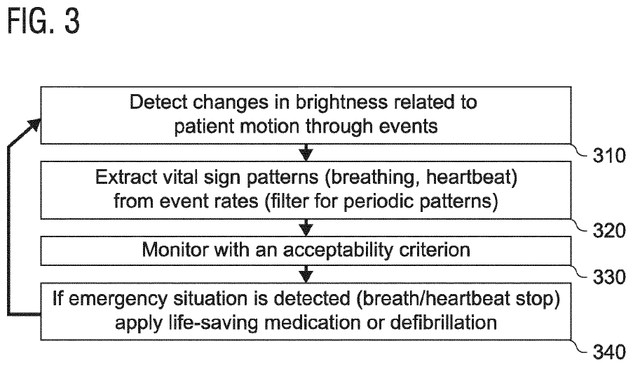

More details and aspects of the apparatus, method, computer program and system are mentioned in connection with the proposed concept, or one or more examples described above or below (e.g., FIGS. 3 to 7). The apparatus, method, computer program and system may comprise one or more additional optional features corresponding to one or more aspects of the proposed concept, or one or more examples described above or below.

Various examples of the present disclosure relate to a concept for macro (and micro) event-based vision sensor patient monitoring, optionally including reacting to the monitoring.

Event-based Vision Sensor (EVS) cameras detect changes in light intensity, with the changes usually relating to moving objects. Patients can be monitored on a macroscopic level (an event camera looking at them from the ceiling), and vital signs can be extracted from the acquired event data. Breathing or heartbeat patterns can be analyzed, and emergency situations can be detected. In case of anomalies, drugs can be automatically dispensed to the patient or defibrillation can be applied. Accordingly, the proposed concept relates to medical equipment.

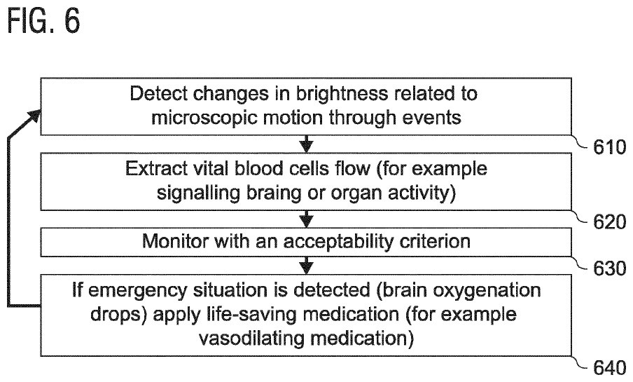

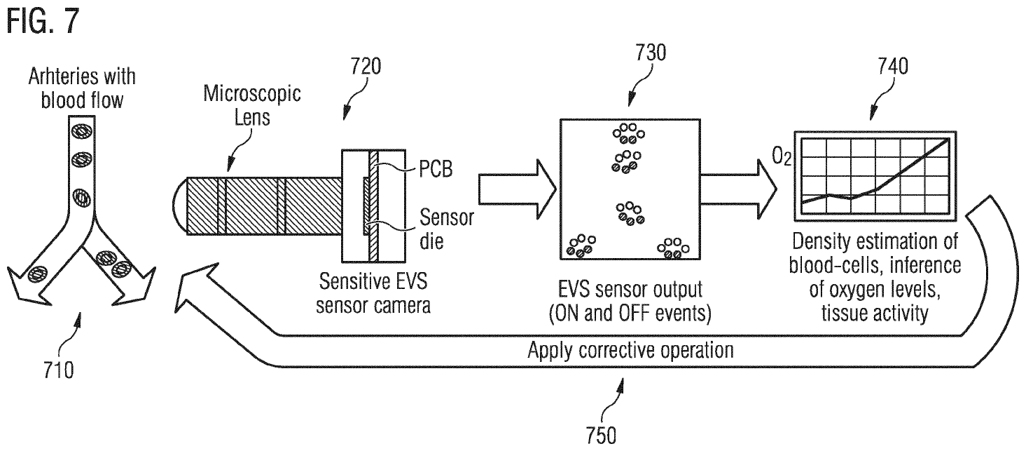

The monitoring can occur at a microscopic level too, as shown in FIGS. 6 and 7, where the EVS looks closely at live tissues and can allow, through a similar processing, the counting of blood-cells arriving through arteries. Since the blood cells signal oxygenation and health of the tissue, corrective measures (such as vasodilating drugs) can also be applied.

The proposed concept may be used to replace or augment current monitoring devices some facilities, e.g., in Hospitals and care homes. For example, breathing and heartbeat monitoring of the patient (vital signs) may be performed to identify and signal an emergency or irregular situation. Some aspects of the proposed concept may also be used in a research environment/during surgeries, e.g., to determine a blood cells flow at microscopic level, which signals or indicates tissue oxygenation levels.

In the proposed concept, frame-based cameras are replaced with Event-based Vision Sensors (EVS) for the previously mentioned scenarios. Algorithms are developed accordingly, which may be capable of automatically releasing drugs or acting in detected emergency situations. EVS are sensors that are capable of continuously extracting changing information in a scene. The extracted information relates to brightness changes or motion directly, reducing the effort required in post-processing significantly. An EVS sensor may be tuned to the task at hand, e.g., to obtain an event signal that is less noisy and/or to be more sensitive to motion, further pushing the applicability of the EVS technology. EVS sensors can be used in an effective closed feedback loop to quickly react to emergency situations, while preserving patient privacy.

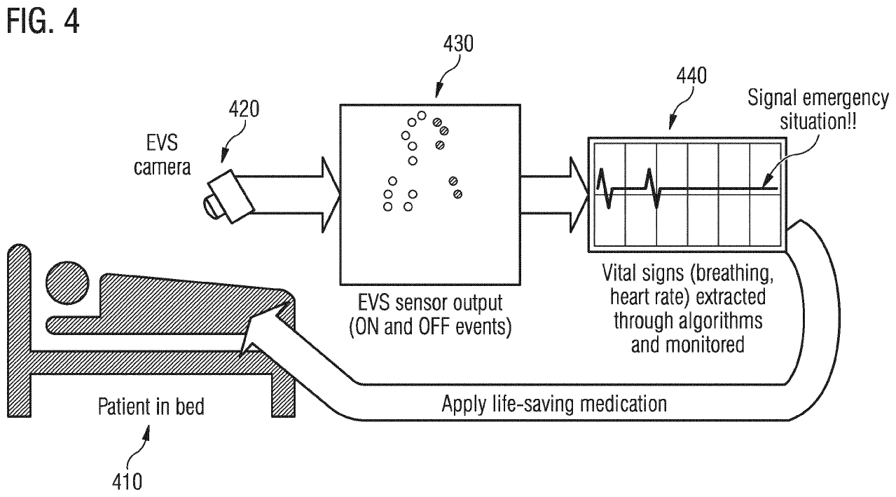

The reduction in post-processing may be applied in hospital and care homes (shown in FIG. 4). Monitoring that uses an EVS sensor can be performed continuously with low power and low data rate from a fixed monitoring point. A large part (or all) of the event data extracted from the scene directly relates to patient motion. During resting periods, the most noticeable motion will mostly be related to breathing or heartbeat. The EVS sensor can also be mounted on a bracelet or close to the neck of the patient on the bed to observe blood pumping activity. Lighting conditions are generally not be an issue thanks to the sensor's High Dynamic Range (HDR). Moreover, privacy concerns may be eliminated since the gathered information is usually not enough to reconstruct faces. If irregular patterns in activity are detected, then emergency actions can be undertaken (such as releasing medication or applying defibrillation). An example of a proposed algorithm can be seen in FIG. 3.

FIG. 3 shows a flow chart of an example of an algorithm for monitoring vital signs (and reacting to emergency situations). The algorithm comprises detecting changes in brightness related to patient motion through events (generated by an event-based vision sensor) 310. The algorithm comprises extracting vital sign patterns (like breathing or heartbeat) from event rates (e.g., using a filter for periodic patterns) 320. The algorithm comprises monitoring the vital sign patterns with an acceptability criterion (or acceptability criteria) 330. If an emergency situation is detected (e.g., if the breathing or heartbeat stops or slows below a threshold), life-saving medication or defibrillation may be applied 340. Subsequently, the algorithm returns to detecting changes in the brightness 310.

FIG. 4 shows a schematic drawing of the application of the algorithm in a care home or hospital. In FIG. 4, a patient 410 is shown sleeping in bed, with an EVS camera 420 being used to generate an event signal 430 based on brightness changes, and thus motion, of the patient 410. The event signal 430, i.e., the EVS sensor output, comprises on and off events, i.e., events indicating that a change in lightness has occurred. From the events signal 430, vital signs 440 (such as breathing or heart rate) are extracted through algorithms and monitored. If an emergency situation is detected during monitoring of the vital signs, a signal is generated in response to the emergency situation, and life-saving medication can be applied to the patient 410, e.g., through a medical intervention device.

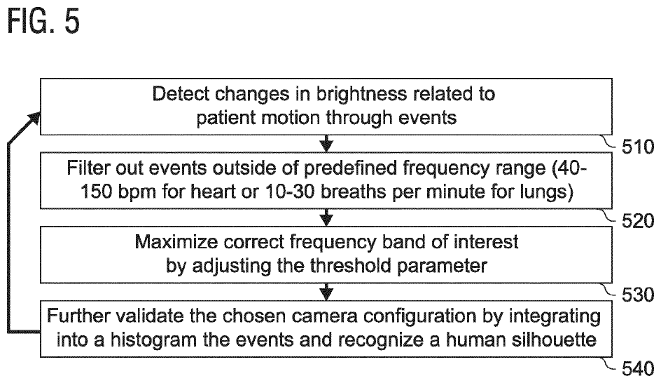

To make sure that the breathing is detected, the sensitivity of the EVS sensor settings can be initialized or periodically adjusted by another algorithm (e.g., as shown in FIG. 5). For example, uncorrelated noise can be filtered out. Events at too low or too high frequency can be filtered out: events which occur with a reasonable rate (40-150 bpm or Hz for the heart or 10-30 breaths per minutes for lungs) can be further processed. In an initial calibration procedure, the event data corresponding to this frequency range can be increased (e.g., boosted or maximized) for a sweep of contrast threshold of the sensor. For too high thresholds (at the EVS sensor), the weak signal might be lost, for too low threshold too much noise would hide the signal. The generated event signal may be integrated into a histogram of a few seconds and can be passed to a (convolutional) neural network trained to recognize a person silhouette. The network can be trained on recognizing people (silhouettes) using an existing frame-based dataset converted using an available EVS simulator to generate events. The sensor parameters maximizing the likelihood of a detection may then be validated or further tweaked.

FIG. 5 shows a flow chart of an example of an algorithm for initial calibration to detect heart or breathing rate. The algorithm starts by detecting changes in brightness related to patient motion through events 510. Events outside of a pre-defined frequency range (40-150 bpm for heart rate or 10-30 breaths per minute for lungs/breathing rate) are filtered out 52-. The frequency band (with respect to events/unit of time) are maximized (i.e., increased or boosted) 530 by adjusting the threshold parameter of the EVS sensor. The chosen camera may be further validated 540 by integrating the events into a histogram and recognizing a human silhouette. Subsequently, the algorithm may return to detecting changes in the brightness 510.

FIG. 6 shows a flow chart of an example of an algorithm for microscopic patient monitoring. The algorithm comprises detecting 610 changes in brightness related to microscopic motion through events. The algorithm comprises extracting 620 vital blood cells flow (for example signaling brain or organ activity. The algorithm comprises monitoring 630 with an acceptability criterion (or criteria). If an emergency situation is detected (e.g., brain oxygenation drops), the algorithm comprises applying 640 life-saving medication (for example vasodilating medication).

FIG. 7 shows a schematic diagram of microscopic monitoring of blood cells in tissue. In this aspect of the present disclosure, live tissue (such as arteries 710 with blood flow) is monitored through microscope lenses of an EVS system 720 comprising a microscopic lens and a sensitive EVS sensor camera (comprising a sensor die that is placed on a printed circuit board, PCB). The resulting EVS sensor output 730 (the event signal) comprises, on and off events, and is used to perform a density estimation 740 of blood-cells, which may allow inference of oxygen levels and tissue activity. Blood cell flow through tiny arteries can be studied to estimate oxygen concentration arriving to the tissue or brain (which is also an indirect sign of neuron activation). Corrective actions 750 (e.g., vasodilator or vasoconstrictor drugs) can be released accordingly to alter abnormal flow and preserve the organ's correct functioning. An EVS sensor tuned to the task at hand can be developed (to be less noisy, more sensitive, etc.) further pushing the applicability of the EVS technology.

More details and aspects of the concept for event-based vision sensor patient monitoring are mentioned in connection with the proposed concept or one or more examples described above or below (e.g., FIG. 1a to 2). The concept for event-based vision sensor patient monitoring may comprise one or more additional optional features corresponding to one or more aspects of the proposed concept, or one or more examples described above or below.

The aspects and features described in relation to a particular one of the previous examples may also be combined with one or more of the further examples to replace an identical or similar feature of that further example or to additionally introduce the features into the further example. Various examples provide:
(1) An apparatus comprising:
   an interface for communicating with an event-based vision sensor; and
   processing circuitry configured to:
   obtain an event signal from the event-based vision sensor, the event signal representing a detected change in luminance detected by the event-based vision sensor,
   determine a motion of at least a body part of a living body based on the event signal, and
   determine at least one vital sign of the living body based on the motion.
(2) The apparatus according to (1), wherein the processing circuitry is configured to determine the at least one vital sign of the living body based on a periodicity of the motion.
(3) The apparatus according to (2), wherein the processing circuitry is configured to determine peaks in the event signal, and to determine the periodicity of the motion based on the peaks in the event signal.
(4) The apparatus according to (3), wherein the processing circuitry is configured to disregard non-periodic motion represented in the event signal.
(5) The apparatus according to one of (3) or (4), wherein the processing circuitry is configured to determine the periodic motion based on the peaks in the event signal and based on a pre-determined range of supported periodicity, the pre-determined range of supported periodicity being based on the vital sign being determined.
(6) The apparatus according to (5), wherein the pre-determined range of supported periodicity imposes a lower limit and an upper limit of periodicities that are characteristic for the vital sign being determined.
(7) The apparatus according to one of (1) to (6), wherein the processing circuitry is configured to determine at least one of a heart rate of the living body and a breathing rate of the living body based on the motion.
(8) The apparatus according to one of (1) to (7), wherein the processing circuitry is configured to aggregate the event signal over a pre-defined time interval, to determine an outline of the living body based on the aggregated event signal, and to determine the at least one vital sign based on the outline of the living body.
(9) The apparatus according to (8), wherein the processing circuitry is configured to disregard a portion of the event signal based on the outline of the living body.
(10) The apparatus according to one of (8) or (9), wherein the living body is a living human body, wherein the processing circuitry is configured to process the outline of the living body using a machine-learning model to determine an identity of the living human body, and to determine the at least one vital sign based on the identity of the living human body.
(11) The apparatus according to (10), wherein the processing circuitry is configured to determine a pre-determined range of supported periodicity of the motion based on the identity of the living human body.
(12) The apparatus according to one of (10) or (11), wherein the processing circuitry is configured to log the at least one vital sign based on the identity of the living human body.
(13) The apparatus according to one of (1) to (12), wherein the processing circuitry is configured to determine the at least one vital sign based on periodic motion exhibited by the living body and/or wherein the processing circuitry is configured to disregard motion having an extent that exceeds a motion threshold.
(14) The apparatus according to one of (1) to (13), wherein the processing circuitry is configured to determine peaks in the event signal, and to adjust a contrast threshold of the event-based vision sensor based on a ratio between the peaks in the event signal and other portions of the event signal.
(15) The apparatus according to (14), wherein the processing circuitry is configured to sweep the contrast threshold, determine peaks in the event signal that are based on the sweep of the contrast threshold, and set a contrast threshold that yields a desired ratio between the peaks in the event signal and other portions of the event signal.
(16) The apparatus according to one of (1) to (15), wherein the processing circuitry is configured to generate an output signal based on the determined at least one vital sign.
(17) The apparatus according to (16), wherein the processing circuitry is configured to generate the output signal if the determined at least one vital sign exceeds an upper threshold or falls below a lower threshold.
(18) The apparatus according to one of (16) or (17), wherein the processing circuitry is configured to control a medical intervention device using the output signal.
(19) The apparatus according to (18), wherein the medical intervention device is a defibrillation device.
(20) The apparatus according to (18), wherein the medical intervention device is a device for controlling a release of medication to the living body.
(21) The apparatus according to one of (16) to (20), wherein the output signal comprises an alarm signal.
(22) A system comprising a camera with an event-based vision sensor and the apparatus according to one of (1) to (21).
(23) The system according to (22), wherein the camera is one of a wall-mounted camera, a ceiling-mounted camera, and a wearable camera.
(24) The system according to one of (22) or (23), further comprising a medical intervention device, wherein the apparatus is configured to control the medical intervention device.
(25) A method comprising:
   obtaining an event signal from an event-based vision sensor, the event signal representing a detected change in luminance detected by the event-based vision sensor;
   determining a motion of at least a body part of a living body based on the event signal; and
   determining at least one vital sign of the living body based on the motion.
(26) A computer program with a program code for performing the method according to (25) when the computer program is executed on a processor.

Examples may further be or relate to a (computer) program including a program code to execute one or more of the above methods when the program is executed on a computer, processor, or other programmable hardware component. Thus, steps, operations, or processes of different ones of the methods described above may also be executed by programmed computers, processors, or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrays ((F) PLAs), (field) programmable gate arrays ((F) PGAs), graphics processor units (GPU), application-specific integrated circuits (ASICs), integrated circuits (ICs) or system-on-a-chip (SoCs) systems programmed to execute the steps of the methods described above.

It is further understood that the disclosure of several steps, processes, operations, or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process, or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

### LIST OF REFERENCES

10 Apparatus
12 Interface
14 Processing circuitry
16 Storage circuitry
100 Computer system
110 Event-based vision sensor
120 Medical monitor
130 Infusion pump
140 Defibrillation device
210 Obtaining an event signal
220 Aggregating the event signal
222 Determining an outline
224 Disregarding a portion of the event signal
226 Determining an identity of the human living body
230 Determining peaks in the event signal
240 Determining a motion
250 Determining a periodicity of the motion
260 Sweeping/adjusting a contrast threshold
270 Determining at least one vital sign
280 Logging the vital sign
290 Generating an output signal
295 Controlling a medical intervention device
310 Detect changes in brightness related to patient motion through events
320 Extract vital sign patterns (breathing, heartbeat) from event rates (filter for periodic patterns)
330 Monitor with an acceptability criterion
340 If an emergency situation is detected (breath/heartbeat stop), apply life-saving medication or defibrillation
410 Patient in bed
420 EVS camera
430 EVS sensor output (event signal)
440 Vital signs
510 Detect changes in brightness related to patient motion through events
520 Filter out events outside of pre-defined frequency range (40-150 bpm for heart or 10-30 breaths per minute for lungs)
530 Maximize correct frequency band of interest by adjusting the threshold parameter
540 Further validate the chosen camera configuration by integrating into a histogram and recognized a human silhouette

What is claimed is:

1. An apparatus comprising:
an interface for communicating with an event-based vision sensor; and
processing circuitry configured to:
obtain an event signal from the event-based vision sensor, the event signal representing a detected change in luminance detected by the event-based vision sensor,
determine a motion of at least a body part of a living body based on the event signal, and
determine at least one vital sign of the living body based on the motion.

2. The apparatus according to claim 1, wherein the processing circuitry is configured to determine the at least one vital sign of the living body based on a periodicity of the motion.

3. The apparatus according to claim 2, wherein the processing circuitry is configured to determine peaks in the event signal, and to determine the periodicity of the motion based on the peaks in the event signal.

4. The apparatus according to claim 3, wherein the processing circuitry is configured to disregard non-periodic motion represented in the event signal.

5. The apparatus according to claim 3, wherein the processing circuitry is configured to determine the periodic motion based on the peaks in the event signal and based on a pre-determined range of supported periodicity, the pre-determined range of supported periodicity being based on the vital sign being determined.

6. The apparatus according to claim 5, wherein the pre-determined range of supported periodicity imposes a lower limit and an upper limit of periodicities that are characteristic for the vital sign being determined.

7. The apparatus according to claim 1, wherein the processing circuitry is configured to determine at least one of a heart rate of the living body and a breathing rate of the living body based on the motion.

8. The apparatus according to claim 1, wherein the processing circuitry is configured to aggregate the event signal over a pre-defined time interval, to determine an outline of the living body based on the aggregated event signal, and to determine the at least one vital sign based on the outline of the living body.

9. The apparatus according to claim 8, wherein the processing circuitry is configured to disregard a portion of the event signal based on the outline of the living body.

10. The apparatus according to claim 8, wherein the living body is a living human body, wherein the processing circuitry is configured to process the outline of the living body using a machine-learning model to determine an identity of the living human body, and to determine the at least one vital sign based on the identity of the living human body.

11. The apparatus according to claim 10, wherein the processing circuitry is configured to determine a pre-determined range of supported periodicity of the motion based on the identity of the living human body.

12. The apparatus according to claim 1, wherein the processing circuitry is configured to determine peaks in the event signal, and to adjust a contrast threshold of the event-based vision sensor based on a ratio between the peaks in the event signal and other portions of the event signal.

13. The apparatus according to claim 12, wherein the processing circuitry is configured to sweep the contrast threshold, determine peaks in the event signal that are based on the sweep of the contrast threshold, and set a contrast threshold that yields a desired ratio between the peaks in the event signal and other portions of the event signal.

14. The apparatus according to claim 1, wherein the processing circuitry is configured to generate an output signal based on the determined at least one vital sign if the determined at least one vital sign exceeds an upper threshold or falls below a lower threshold.

15. The apparatus according to claim 14, wherein the processing circuitry is configured to control a medical intervention device using the output signal.

16. The apparatus according to claim 15, wherein the medical intervention device is a defibrillation device or a device for controlling a release of medication to the living body.

17. A system comprising a camera with an event-based vision sensor and the apparatus according to claim 1.

18. The system according to claim 17, wherein the camera is one of a wall-mounted camera, a ceiling-mounted camera, and a wearable camera.

19. A computer-implemented method comprising:
obtaining an event signal from an event-based vision sensor, the event signal representing a detected change in luminance detected by the event-based vision sensor;
determining a motion of at least a body part of a living body based on the event signal; and
determining at least one vital sign of the living body based on the motion.

20. A computer program with a program code for performing the method according to claim 19 when the computer program is executed on a processor.
